(19) United States
(12) Patent Application Publication
(10) Pub. No.: US 2026/0191569 A1
(43) Pub. Date: Jul. 9, 2026

(54) A DYNAMIC INTRAMEDULLARY NAIL WITH A HYBRID DRIVE; A DYNAMIC INTRAMEDULLARY NAIL SET WITH A MAGNETIC ACTUATOR

(71) Applicant: ORTHOGET SPOLKA Z OGRANICZONA ODPOWIEDZIALNOSCIA, Wroclaw (PL)

(72) Inventors: Piotr KOWALEWSKI, Swidnica (PL); Dominika GRYGIER, Jelenia Góra (PL); Mariusz OPALKA, Wroclaw (PL); Jakub SLOWINSKI, Wroclaw (PL); Mateusz DZIUBEK, Wroclaw (PL); Robert JASTRZABEK, Poznan (PL); Pawel KROWICKI, Wroclaw (PL)

(73) Assignee: ORTHOGET SPOLKA Z OGRANICZONA ODPOWIEDZIALNOSCIA, Wroclaw (PL)

(21) Appl. No.: 18/868,004

(22) PCT Filed: May 25, 2023
(86) PCT No.: PCT/PL2023/050038

§ 371 Date: Nov. 21, 2024

(30) Foreign Application Priority Data
May 25, 2022 (PL) P.441270

(51) Int. Cl. A61B 17/72

(52) U.S. Cl. CPC A61B 17/7216

(57) ABSTRACT

The object of the invention is a dynamic intramedullary nail with a hybrid drive, characterised in that it comprises a casing consisting of a proximal and a distal segment, and the casing has comprised therein a magnetic rotor for inducing an external magnetic field, and an inner spring element cumulating potential energy during assembly, constituting driving means for lengthening the nail in a telescopic manner.

The object of the invention is an intramedullary nail with a variable length, driven by means of a hybrid drive system. The implant allows for correcting the length of the bone being repaired. Lengthening or shortening of the nail allows for controlling the speed of growth and repair of the bone. The hybrid drive used in the implant allows for achieving the required force during extension, and for complete control of the nail length by means of an external device.

One of the methods for solving the problems related to the unevenness of limbs is their surgical lengthening. The lengthening of limbs by means of external stabilisers is cumbersome and dangerous for the patient, due to their size and great chances of infection and other complications. The use of an internal mechanism which is the intramedullary nail causes the healing process to proceed painlessly, while the inner implant does not hinder the patient's everyday functioning. Moreover, there is a much lower risk of wound infection.

The existing solutions of dynamic intramedullary nails are characterised by a single source of energy driving the elements of the implant. A dynamic intramedullary nail with a hybrid drive uses the mechanical energy cumulated in a resilient element located inside the implant, as well as magnetic energy originating from the outside in a synchronised manner. The magnetic drive allows for introducing energy from an external actuator to the device through the patient's tissues. The magnetic energy is converted inside the implant into mechanical energy moving the mechanism. At the same time, the mechanical energy cumulated in the resilient element, with specific characteristics, supports the working motion of the implant during the lengthening process. The force caused by the resilient element compensates for the mechanical interactions generated in soft tissues (muscles, tendons, and skin), therefore relieving the magnetic drive.

The combination of two types of drives both allows for controlling the lengthening process during healing, and minimises the amount of energy generated outside and passing through the patient's body. It also minimises the size of the magnetic drive inside the implant, improving the compactness and mechanical strength of the structure. The strength of the magnetic field generated by the external element driving the nail mechanism is limited to an absolute minimum.

From Polish patent description no. PL162850B1 there is a known bone repair device intended for correcting bones undergoing repair in a lengthwise direction, with play within specified limits and correction in the transverse direction. The device has a fixed and a movable jaw, connected by a bolt. The jaws have guide channels and oval openings for mounting bone implants. The channels and openings are perpendicular to a plane passing through the lengthwise axis of a cylindrical guide.

From Polish patent description PL226930B1 there is a known expandable intramedullary nail for bone lengthening, with a piezoelectric mechanism, which is intended for bone lengthening, with a lengthening mechanism based on piezoelectric actuators. The nail consists of a middle body, an upper body with a transverse opening and a lengthwise opening, and a pin, on which bellows and a piezoelectric mechanism mounted to the flat surface of a lower slot are situated, while the middle body has an upper and lower slot with ratcheting elements extendable to the outside and introduced into grooves in the middle body, the ratcheting elements being based on a spring element which has an opening and 12 openings on the whole periphery.

From Polish patent description PL237952B1 there is a known dynamic intramedullary nail enabling angular correction, which is driven by an external magnetic field, and in its construction it has rotary rings with skewed front surfaces, whose rotation causes the nail to bend, which provides the possibility of changing the angular orientation of the ends of the bone being repaired. The construction of the nail is based on a telescopic extending mechanism with the ability to independently rotate one of the ends of the nail, and the ability to bend the nail. The drive of the nail lengthening and bending mechanism constitutes a magnetic transducer placed in the middle part of the nail. Due to the effect of an external magnetic field, the magnetic transducer rotates and drives both nail mechanisms independently.

The device for lengthening long bones known from patent description U.S. Pat. No. 5,961,553B has a lengthening mechanism based on an electric motor, which rotates the guide screw of a linear actuator. The electric motor is connected to the guide screw via a reduction gear. The part of the implant comprising a rope drive and a piston are attached to the bone. The value of lengthening is determined based on the measurement of the number of revolutions of the guide screw. The system measuring the number of revolutions of the guide screw also identifies the direction of the revolutions.

From patent description U.S. Pat. No. 5,415,660 there is a known implantable limb lengthening nail driven by a shape memory alloy, whose lengthening mechanism is based on a shape memory alloy and a ratcheting assembly. The implant is formed from two telescoping cylinders which are connected to the bone. The element made of the alloy moves a rod having edges adjusted to engage the hooks of the ratcheting assembly. The edges have the shape of ledges narrowing gradually in one direction. This makes the movement of the rod possible in one direction only.

From patent description U.S. Pat. No. 5,976,138B there is a known distraction system for long bones, which displaces the middle fragment of a bone divided into three fragments (proximal, intermediate, and distal). The proximal and distal fragments are connected to the casing of the implant, while the intermediate fragment can be displaced. The implant is provided with a linear actuator based on an internal drive, which rotates a guide screw. The implant is attached to the bone fragments by means of screws.

The implant known from patent description US2014/0236311A1 has the form of a linear actuator, whose two elements have been attached to two bone fragments. The implant replaces a removed bone fragment. The drive constitutes a piezoelectric motor connected to a gear driving the guide screw. The rotary motion of the guide screw is converted into the linear motion of a shaft by means of lengthwise grooves and screws placed in the casing and entering the grooves. The drive is powered inductively from an external source.

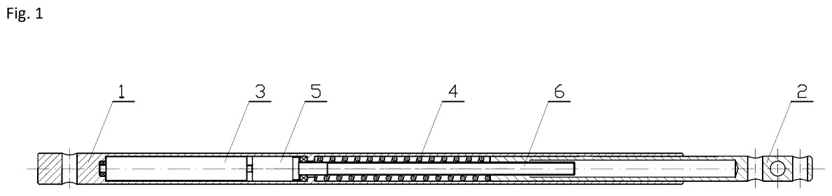

The object of the invention is presented in more detail in the drawing, in which FIG. 1 presents a construction schematic of the dynamic intramedullary nail with a hybrid drive.

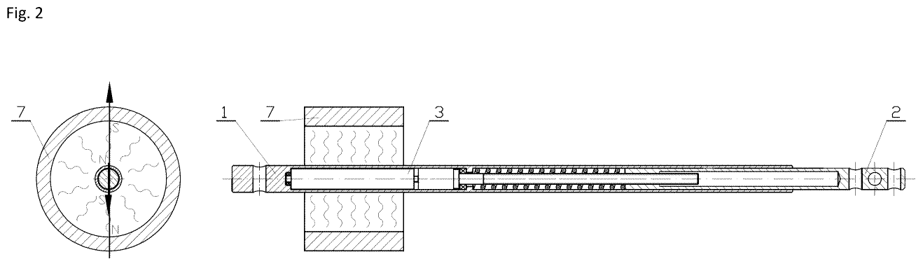

FIG. 2 presents the intramedullary nail with a hybrid drive along with an external actuator and the impact of a magnetic field.

The essence of the dynamic intramedullary nail with a hybrid drive according to the invention is that it has a construction based on a telescopic mechanism, which enables lengthening or shortening the implant.

The dynamic intramedullary nail (implant) consists of a skeleton (shell) and a drive located inside the shell. The skeleton of the nail is made of: a proximal segment (1), having a first end for mounting in the bone and a second end, and a distal segment (2), having a first end for mounting in the bone and a second end. The second ends of both members are connected telescopically. The hybrid drive of the lengthening mechanism of the nail constitutes a simultaneously acting magnetic rotor (3) and spring element (4), which are placed in the central part of the nail. Due to the action of an external magnetic field generated from an external actuator (7), the magnetic rotor (3) rotates and drives a mechanical gear (5). The mechanical gear (5) drives the driving screw (6), which causes extension of the nail by being driven out of the distant segment (2). The actuator (7) may have a varying construction. For example, it may be based on electromagnets with a variable field, or use rotating permanent magnets. The actuator (7) is a separate construction. It is important for it to be able to generate a variable magnetic field, so as to cause rotation of the magnetic rotor.

The construction of the dynamic intramedullary nail with a hybrid drive enables the drive to simultaneously use forces originating from the spring element closed inside the implant, and forces originating from an external source of energy—via a magnetic field.

The dynamic intramedullary nail has in its construction a magnetic rotor (3) being a permanent magnet, which is moved by magnetic engagement by the external actuator (7). The movement of the magnetic rotor (3) via additional elements of the drive system—the mechanical gear (5) and the driving screw (6)—causes adjustment of extension and retraction of the elements of the implant, and therefore a change in its length. The direction of movement of the external actuator (7) decides about lengthening or shortening the implant. The mechanical gear (5) comprises three assemblies of gear wheels, each consisting of three wheels operating in a rotary motion—planetary with respect to the central wheel. The rotation of the shackle on which the planetary wheels are mounted drives the central wheel of the next set of wheels. The last shackle drives the driving screw. Inside the intramedullary nail with a hybrid drive, there is also the spring element (4), which becomes deformed during assembly (the nail production process); this potential energy cumulated therein supports the nail extension motion. The spring element (4) can be for example selected among: a coil spring, a spiral spring, a sheet spring, and a gas spring. The essence of the spring element is to accumulate potential energy. The force generated by the spring element (4) reduces the reaction of soft tissues of the limb, increasing the efficiency of the implant. The magnetic system consisting of the external magnetic actuator (7) and the internal magnetic rotor (3) enables controlling the direction and speed of extension of the nail. The nail may be used to stabilise the bone after injuries, and for osteodistraction after an osteotomy.

1. A dynamic intramedullary nail with a hybrid drive, characterised in that it comprises a casing consisting of a proximal and a distal segment, and the casing has comprised therein a magnetic rotor driven by an external magnetic field, and an inner spring element accumulating potential energy during assembly, constituting driving means for lengthening the nail in a telescopic manner.

2. The dynamic intramedullary nail with a hybrid drive according to claim 1, characterised in that the proximal segment comprises a first end for mounting in a bone, and a second end.

3. The dynamic intramedullary nail with a hybrid drive according to claim 1, characterised in that the distal segment comprises a first end for mounting in a bone, and a second end.

4. The dynamic intramedullary nail with a hybrid drive according to claim 1, characterised in that the second end of the proximal segment is connected telescopically with the second end of the distal segment.

5. The dynamic intramedullary nail with a hybrid drive according to claim 1, characterised in that a driving screw extends from the proximal segment to the distal segment, the screw having a first end comprising a mechanical gear, and the second end of the screw being seated in the second end of the distal segment.

6. The dynamic intramedullary nail with a hybrid drive according to claim 1, characterised in that the magnetic rotor is actuated by an external magnetic field generated by an external magnetic actuator.

7. The dynamic intramedullary nail with a hybrid drive according to claim 1, characterised in that the driving screw extends lengthwise through the spring element.

8. The dynamic intramedullary nail with a hybrid drive according to claim 1, characterised in that the driving means allow for lengthening or shortening the length of the intramedullary nail depending on the direction of changes in the external magnetic field, simultaneously using the potential energy cumulated in the internal spring element.

9. The dynamic intramedullary nail with a hybrid drive according to claim 1, characterised in that it has a magnetic rotor moved by an external magnetic field originating from the magnetic actuator and converted into the motion of the nail, simultaneously using the forces supporting this extension, originating from the element placed inside the implant.

10. The dynamic intramedullary nail with a hybrid drive according to claim 1, characterised in that the mechanical gear constitutes a planetary gearset.

11. A set comprising the dynamic intramedullary nail with a hybrid drive according to claim 1, and a magnetic actuator for use in osteodistraction procedures.

12. The dynamic intramedullary nail with a hybrid drive according to claim 2, characterised in that the second end of the proximal segment is connected telescopically with the second end of the distal segment.

13. The dynamic intramedullary nail with a hybrid drive according to claim 3, characterised in that the second end of the proximal segment is connected telescopically with the second end of the distal segment.

14. The dynamic intramedullary nail with a hybrid drive according to claim 3, characterised in that a driving screw extends from the proximal segment to the distal segment, the screw having a first end comprising a mechanical gear, and the second end of the screw being seated in the second end of the distal segment.

15. The dynamic intramedullary nail with a hybrid drive according to claim 5, characterised in that the driving screw extends lengthwise through the spring element.
